(19) United States
(12) Patent Application Publication
(10) Pub. No.: US 2022/0162706 A1
(43) Pub. Date: May 26, 2022

(54) INTRA-PATIENT GENOMIC HETEROGENEITY OF SINGLE CIRCULATING TUMOR CELLS (CTCS) ASSOCIATED TO PHENOTYPIC CTC HETEROGENEITY IN METASTATIC CASTRATE RESISTANT PROSTATE CANCER (MCRPC)

(71) Applicant: Epic Sciences, Inc., San Diego, CA (US)

(72) Inventors: Ryan Dittamore, San Diego, CA (US); Mark Landers, Carlsbad, CA (US)

(21) Appl. No.: 17/377,074

(22) Filed: Jul. 15, 2021

Related U.S. Application Data
(62) Division of application No. 15/577,307, filed on Nov. 27, 2017, now abandoned.
(60) Provisional application No. 62/168,607, filed on May 29, 2015.

(51) Int. Cl. C12Q 1/6886; G01N 33/52; G01N 33/574

(52) U.S. Cl. CPC C12Q 1/6886; G01N 33/52; C12Q 2600/156; C12Q 2600/112; G01N 33/57434

(57) ABSTRACT

The disclosure provides methods correlating intra-patient genomic heterogeneity of single CTCs with phenotypic heterogeneity in each of a population of prostate cancer (PCa) patients.

This application is a divisional of U.S. patent application Ser. No. 15/577,307, filed Nov. 27, 2017, which is a national stage application of International Patent Application No. PCT/US2016/034640, filed May 27, 2016, which claims the benefit of priority of U.S. Provisional Application No. 62/168,607, filed May 29, 2015, the entire contents of each of which are incorporated herein by reference.

The present disclosure relates generally to methods for correlating observed CTC phenotypic profiles and genomic profiles in CTC subpopulations associated with metastatic castration-resistant prostate cancer (mCRPC).

### BACKGROUND

Prostate cancer is the most commonly diagnosed solid organ malignancy in the United States (US) and remains the second leading cause of cancer deaths among American men. In 2014 alone, the projected incidence of prostate cancer is 233,000 cases with deaths occurring in 29,480 men, making metastatic prostate cancer therapy truly an unmet medical need. Siegel et al., 2014. CA Cancer J Clin. 2014; 64(1):9-29. Epidemiological studies from Europe show comparable data with an estimated incidence of 416700 new cases in 2012, representing 22.8% of cancer diagnoses in men. In total, 92200 PC-specific deaths are expected, making it one of the three cancers men are most likely to die from, with a mortality rate of 9.5%

Despite the proven success of hormonal therapy for prostate cancer using chemical or surgical castration, most patients eventually will progress to a phase of the disease that is metastatic and shows resistance to further hormonal manipulation. This has been termed metastatic castration-resistant prostate cancer (mCRPC). Despite this designation, however, there is evidence that androgen receptor (AR)-mediated signaling and gene expression can persist in mCRPC, even in the face of castrate levels of androgen. This may be due in part to the upregulation of enzymes involved in androgen synthesis, the overexpression of AR, or the emergence of mutant ARs with promiscuous recognition of various steroidal ligands. Treatment of patients with mCRPC remains a significant clinical challenge.

Prior to 2004, there was no treatment proven to improve survival for men with mCRPC. The treatment of patients with mitoxantrone with prednisone or hydrocortisone was aimed only at alleviating pain and improving quality of life, but there was no benefit in terms of overall survival (OS). In 2004, the results of two major phase 3 clinical trials, TAX 327 and SWOG (Southwest Oncology Group) 9916, established Taxotere® (docetaxel) as a primary chemotherapeutic option for patients with mCRPC. Additional hormonal treatment with androgen receptor (AR) targeted therapies, chemotherapy, combination therapies, and immunotherapy, have been investigated for mCRPC, and recent results have offered additional options in this difficult-to-treat patient group. With the advent of exponential growth of novel agents tested and approved for the treatment of patients with metastatic castration-resistant prostate cancer (mCRPC) in the last 5 years alone, issues regarding the optimal sequencing or combination of these agents have arisen. Several guidelines exist that help direct clinicians as to the best sequencing approach and most would evaluate presence or lack of symptoms, performance status, as well as burden of disease to help determine the best sequencing for these agents. Mohler et al., 2014, J Natl Compr Canc Netw. 2013; 11(12):1471-1479; Cookson et al., 2013, J Urol. 2013; 190(2):429-438. Currently, approved treatments consist of taxane-class cytotoxic agents such as Taxotere® (docetaxel) and Jevtana® (cabazitaxel), and anti-androgen hormonal therapy drugs such as Zytiga® (arbiterone, blocks androgen production) or Xtandi® (enzalutamide, an androgen receptor (AR) inhibitor).

The challenge for clinicians is to decide the best sequence for administering these therapies to provide the greatest benefit to patients. However, therapy failure remains a significant challenge based on heterogeneous responses to therapies across patients and in light of cross-resistance from each agent. Mezynski et al., Ann Oncol. 2012; 23(11):2943-2947; Noonan et al., Ann Oncol. 2013; 24(7):1802-1807; Pezaro et al., Eur Urol. 2014, 66(3): 459-465. In addition, patients may lose the therapeutic window to gain substantial benefit from each drug that has been proven to provide overall survival gains. Hence, better methods of identifying the target populations who have the most potential to benefit from targeted therapies remain an important goal. Analysis of somatic genomic alterations in primary tumors is often used to define mutational status and guide therapeutic decisions. Selective pressures, including multiple lines of therapy, can lead to tumor evolution through step-wise accumulation of genomic alterations.

Circulating tumor cells (CTCs) represent a significant advance in cancer diagnosis made even more attractive by their non-invasive measurement. Cristofanilli et al., N Engl J Med 2004, 351:781-91. CTCs released from either a primary tumor or its metastatic sites hold important information about the biology of the tumor. Historically, the extremely low levels of CTCs in the bloodstream combined with their unknown phenotype has significantly impeded their detection and limited their clinical utility. A variety of technologies have recently emerged for detection, isolation and characterization of CTCs in order to utilize their information. CTCs have the potential to provide a non-invasive means of assessing progressive cancers in real time during therapy, and further, to help direct therapy by monitoring phenotypic physiological and genetic changes that occur in response to therapy. In most advanced prostate cancer patients, the primary tumor has been removed, and CTCs are expected to consist of cells shed from metastases, providing a “liquid biopsy.” While CTCs are traditionally defined as EpCAM/cytokeratin positive (CK+) cells, CD45−, and morphologically distinct, recent evidence suggests that other populations of CTC candidates exist including cells that are EpCAM/cytokeratin negative (CK−) or cells smaller in size than traditional CTCs. These findings regarding the heterogeneity of the CTC population, suggest that enrichment-free CTC platforms are favorable over positive selection techniques that isolate CTCs based on size, density, or EpCAM positivity that are prone to miss important CTC subpopulations.

CTCs from mCRPC patients have shown phenotypic heterogeneity in size, shape, CK expression and Androgen Receptor (AR) expression. Heterogeneity increases with multiple lines of therapy and is associated with treatment resistance. A need exists to define CTC genotype to phenotype correlations that enable identification of emerging resistant clones for which a change in therapy may be needed. The present invention addresses this need and provides related advantages.

### SUMMARY

Disclosed herein is a method for correlating genomic heterogeneity of single CTCs with phenotypic heterogeneity in each of a population of prostate cancer (PCa) patients comprising: (a) performing a direct analysis comprising immunofluorescent staining and morphological characterization of nucleated cells in a blood sample obtained from the patient to identify and enumerate circulating tumor cells (CTC); (b) isolating the CTCs from said sample; (c) individually characterizing genomic alterations and phenotypic features to generate a profile for each of the CTCs; (d) correlating individual genomic heterogeneity of single CTCs with phenotypic heterogeneity in each of the population of PCa patients, and (e) analyzing said correlations of individual genomic heterogeneity of single CTCs with phenotypic heterogeneity across the population of PCa patients to identify a universal correlation of individual genomic heterogeneity of single CTCs with phenotypic heterogeneity. The universal correlation can be utilized to identify one or more phenotypic profiles that correspond to a genotypic profile, thereby the need for characterizing said genomic alterations. Also disclosed are methods of predicting clinical course of PCa, for example, resistance to a particular PCa therapy, based on the genotypic profile.

### BRIEF DESCRIPTION OF THE DRAWINGS

FIGS. 1A through 1C show work flow for sample preparation of the Epic Platform & Copy number variation (CNV) Next Generation Sequencing (NGS), CTC enumeration/characterization and CNV analysis by NGS.

FIGS. 2A and 2B show the experimental design. FIG. 2A describes the mCRPC patient cohort analyzed in this study segregated by treatment (Taxane or AR Therapy) and line of therapy. FIG. 2B shows a table that lists the samples tested, molecular markers tested by IF, the number of CTC/mL detected and number of CTCs sequenced.

FIGS. 3A through 3D show CNV alterations detected and the relationship to CTC phenotype. FIG. 3A shows a histogram summarizing the number of CNV events observed across all 1M bp windows/CTC in all CTCs analyzed. FIG. 3B is a bar chart comparing the number of CNV alterations occurring in windows containing prostate specific tumor genes (n=89). FIG. 3C is a heat map that compares the frequency of copy number alterations (columns), amplifications (green) and deletions (red), for each CTC analyzed (row) unsupervised clustered by genome wide CNV profile and color coded by one of fifteen observed CTC phenotypes. Phenotype characteristics are shown in the lower left panel, describing AR, CK and cell size characteristics for each of the 15 phenotypes. FIG. 3D shows a correlation matrix describing the significant correlations of observed CTC phenotypic features with prostate and tumor specific CNV alterations (n=37). Both positive (blue) and negative correlations between CNV events are compared.

FIGS. 4A through 4C demonstrate the observed intra-patient CTC Heterogeneity. Intra-patient genomic and phenotypic CTC heterogeneity were observed across most patients. The dot plot (FIG. 4A) shows the number of observed CNV alterations for each CTC within a single patient (2nd line, samples 15, 8, 12, 13; 3rd line, samples 16, 17, 11, 3, 1, 9; beyond 3rd line, samples 14, 2, 5, 4, 7, 6, 10). The table (FIG. 4B) further describes the heterogeneity of prominent therapeutic resistance CNV alterations within each patient. Patients are sorted based on line of therapy with either Taxane chemotherapy or ARTx targeted therapies in 2^{nd} line, 3^{rd} line, 4^{th} line and beyond settings. FIG. 4C shows individual examples of CNV profiles hierarchical clustered by genomic profile within 2 separate patients (1 patient responding to therapy, and 1 patient resisting therapy) across all genomic regions analyzed. Images are located to the left of each cell CNV plot.

### DETAILED DESCRIPTION

The present disclosure is based, in part, on the unexpected discovery that intra-patient genomic CTC heterogeneity correlates to phenotypic CTC heterogeneity such that CTC genomic profiles correlate to observed CTC phenotypic profiles. Genotypic and phenotypic heterogeneity demonstrate a linear correlation. As disclosed herein, an average of 8 copy number variation (CNV) alterations can be detected in CTCs of a mCRPC patient and many of the commonly altered CNV windows contain therapeutic relevant gene targets. CTC genomic profiles correlate to observed CTC phenotypic profiles. As further disclosed herein, specific CNV alterations can further be associated with specific copy number gain or loss.

As further described herein, intra-patient genomic CTC heterogeneity can be observed in mCRPC patients, including multiple distinct clonal populations with large variation in number of CNV alterations and detection of CNVs in subpopulations of CTCs that cannot be detected in CTC pools. As further disclosed herein, larger heterogeneity of clonal populations with CNV alterations can be observed in windows containing genes associated with therapeutic resistance in mCRPC patients.

In one embodiment, the present disclosure provides a method for correlating intra-patient genomic heterogeneity of single CTCs with phenotypic heterogeneity in a prostate cancer (PCa) patient comprising:(a) performing a direct analysis comprising immunofluorescent staining and morphological characterization of nucleated cells in a blood sample obtained from the patient to identify and enumerate circulating tumor cells (CTC); (b) isolating the CTCs from said sample; (c) individually characterizing genomic alterations and phenotypic features to generate a profile for each of the CTCs, and (d) correlating genomic heterogeneity of single CTCs with phenotypic heterogeneity in the PCa patient.

In another embodiment, the present disclosure provides a method for correlating intra-patient genomic heterogeneity of single CTCs with phenotypic heterogeneity in each of a population of prostate cancer (PCa) patients comprising: (a) performing a direct analysis comprising immunofluorescent staining and morphological characterization of nucleated cells in a blood sample obtained from the patient to identify and enumerate circulating tumor cells (CTC); (b) isolating the CTCs from said sample; (c) individually characterizing genomic alterations and phenotypic features to generate a profile for each of the CTCs; (d) correlating individual genomic heterogeneity of single CTCs with phenotypic heterogeneity in each of the population of PCa patients, and (e) analyzing said correlations of individual genomic heterogeneity of single CTCs with phenotypic heterogeneity across the population of PCa patients to identify a universal correlation of individual genomic heterogeneity of single CTCs with phenotypic heterogeneity. In this embodiment, a individuals in population of PCa patients can be similarly situated with regard to one or more patient demographics, including, for example, therapy or line of therapy. In some embodiments, the therapy is hormone directed therapy or chemotherapy. In particular embodiments, the hormone directed therapy comprises Androgen Deprivation Therapy (ADT). In some embodiments, the ADT is a second line hormonal therapy including, for example, a therapy that blocks synthesis of androgen or inhibits Androgen Receptor (AR). In some embodiments, the second line hormonal therapy is selected from the group consisting of abiraterone acetate, ketoconazole and aminoglutethimide. In other embodiments, the chemotherapy is taxane therapy.

In some embodiments, the immunofluorescent staining of nucleated cells comprises pan cytokeratin, cluster of differentiation (CD) 45, diamidino-2-phenylindole (DAPI) and androgen receptor (AR).

In some embodiments, the morphological characterization comprises determination of one or more of the group consisting of nucleus size, nucleus shape, presence of holes in nucleus, cell size, cell shape and nuclear to cytoplasmic ratio, nuclear detail, nuclear contour, prevalence of nucleoli, quality of cytoplasm and quantity of cytoplasm.

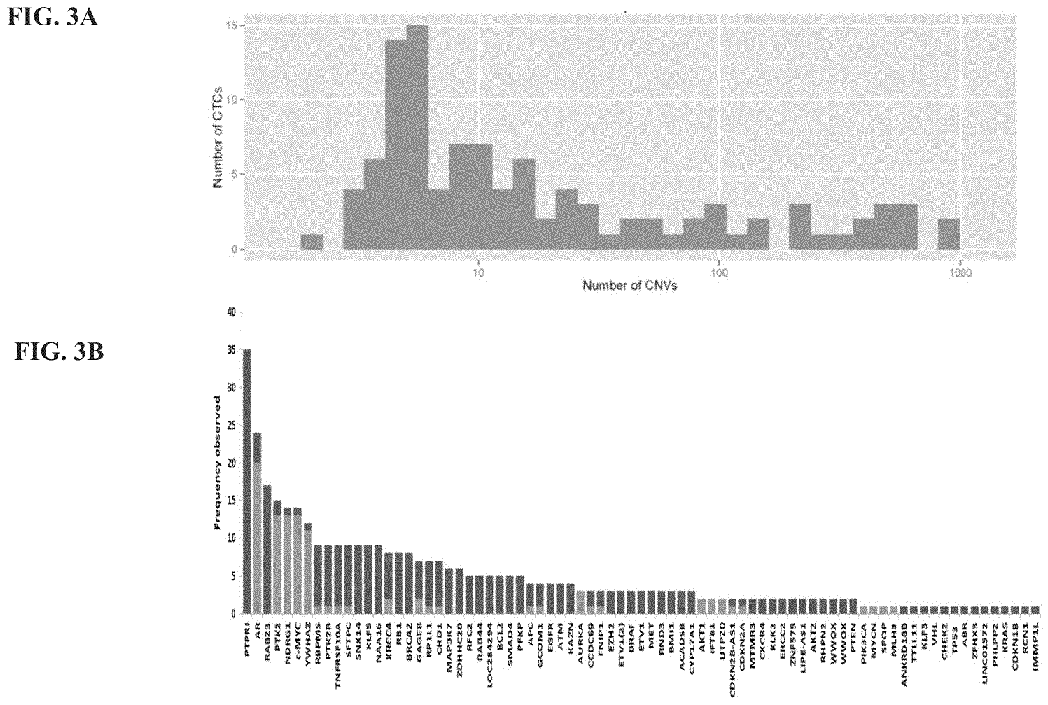

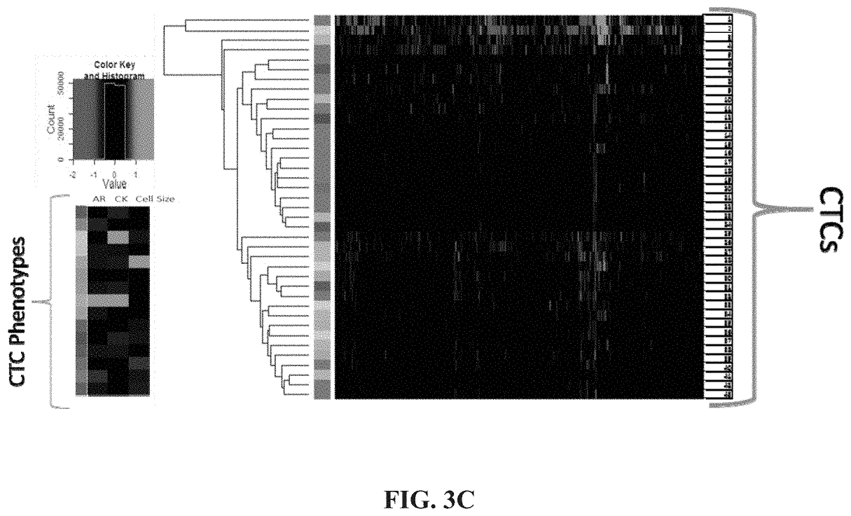

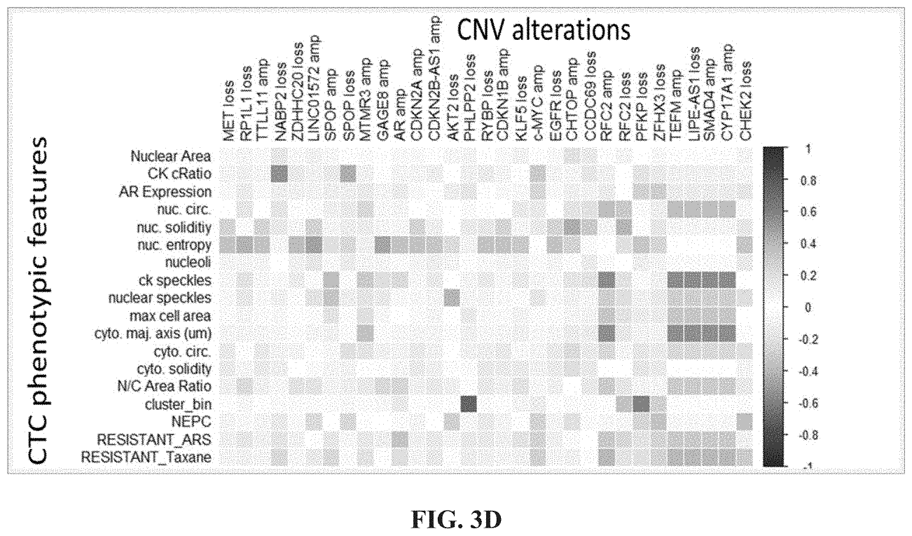

In some embodiments, the phenotypic features of a CTC, including, for example, phenotypic features selected from the group listed in FIG. 3D. In related embodiments, the genomic alterations are copy number variation (CNV) alterations, including, for example, CNV alterations selected from the group listed in FIG. 3D.

In some embodiments, a universal correlation of individual genomic heterogeneity of single CTCs with phenotypic heterogeneity is used to identify a phenotypic profile that corresponds to a genotypic profile. In some embodiments, the identification of said phenotypic profile obviates the need for characterizing said genomic alterations. In particular embodiments, the phenotypic profile is capable of predicting resistance to a PCa therapy, for example, hormone directed therapy or chemotherapy.

A person skilled in the art will appreciate that a number of methods can be used to determine the presence or absence of a biomarker, including microscopy based approaches, including fluorescence scanning microscopy (see, e.g., Marrinucci D. et al., 2012, Phys. Biol. 9 016003).

As used herein, the term “circulating tumor cell” or “CTC” is meant to encompass any rare cell that is present in a biological sample and that is related to prostate cancer. CTCs, which can be present as single cells or in clusters of CTCs, are often epithelial cells shed from solid tumors found in very low concentrations in the circulation of patients. CTCs include “traditional CTCs,” which are cytokeratin positive (CK+), CD45 negative (CD−), contain a DAPI nucleus, and are morphologically distinct from surrounding white blood cells. The term also encompasses “non-traditional CTCs” which differ from a traditional CTC in at least one characteristic. Non-traditional CTCs include the five CTC subpopulations, including CTC clusters, CK negative (CK⁻) CTCs that are positive at least one additional biomarker that allows classification as a CTC, small CTCs, nucleoli⁺CTCs and CK speckled CTCs. As used herein, the term “CTC cluster” means two or more CTCs with touching cell membranes.

As used herein, the term “direct analysis” means that the CTCs are detected in the context of all surrounding nucleated cells present in the sample as opposed to after enrichment of the sample for CTCs prior to detection. In some embodiments, the methods comprise microscopy providing a field of view that includes both CTCs and at least 200 surrounding white blood cells (WBCs).

A fundamental aspect of the present disclosure is the unparalleled robustness of the disclosed methods with regard to the detection of CTCs. The rare event detection disclosed herein with regard to CTCs is based on a direct analysis, i.e. non-enriched, of a population that encompasses the identification of rare events in the context of the surrounding non-rare events. Identification of the rare events according to the disclosed methods inherently identifies the surrounding events as non-rare events. Taking into account the surrounding non-rare events and determining the averages for non-rare events, for example, average cell size of non-rare events, allows for calibration of the detection method by removing noise. The result is a robustness of the disclosed methods that cannot be achieved with methods that are not based on direct analysis, but that instead compare enriched populations with inherently distorted contextual comparisons of rare events. The robustness of the direct analysis methods disclosed herein enables characterization of CTCs, including subpopulations of CTCs described herein, that cannot be achieved with other, enrichment-dependent CTC detection methods and that enables the identification and analysis of morphological and protein biomarkers indicative of the presence of a CTC subpopulation associated with CRPC in the context of the claimed methods. Approaches that enrich CTCs based on epithelial expression or physical characteristics are likely to miss non-traditional CTCs. Enumeration and characterization of non-traditional CTCs in mCRPC and other cancers provides prognostic/predictive information beyond traditional CTCs.

The majority of patients with systemic prostate cancer treated with androgen deprivation therapy (ADT), also referred to a “primary” hormone therapy in the context of prostate cancer, will develop castration-resistant prostate cancer (CRPC). Castration-resistant prostate cancer (CRCP) is defined by disease progression despite androgen deprivation therapy (ADT). CRPC can be categorized as nonmetastatic or metastatic (mCRPC). mCRPC refers to CRPC that has spread beyond the prostate gland to a distant site, such as lymph nodes or bone. The progression of CRCP can encompass as any combination of a rise in serum prostate-specific antigen (PSA), progression of pre-existing disease, and appearance of initial or new metastases. Most CRPCs select mechanisms that upregulate intracellular androgens and/or androgen receptor (AR), leading to ongoing AR-directed cancer growth despite a castrate level of serum androgens. Thus, when patients develop CRPC they are usually sensitive to sequential “secondary” hormonal therapies (antiandrogens, ketoconazole, estrogens) directed at AR inhibition.

A sample can comprise a bodily fluid such as blood; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint; cells; skin, and the like. A biological sample obtained from a subject can be any sample that contains nucleated cells and encompasses any material in which CTCs can be detected. A sample can be, for example, whole blood, plasma, saliva or other bodily fluid or tissue that contains cells.

In particular embodiments, the biological sample is a blood sample. As described herein, a sample can be whole blood, more preferably peripheral blood or a peripheral blood cell fraction. As will be appreciated by those skilled in the art, a blood sample can include any fraction or component of blood, without limitation, T-cells, monocytes, neutrophiles, erythrocytes, platelets and microvesicles such as exosomes and exosome-like vesicles. In the context of this disclosure, blood cells included in a blood sample encompass any nucleated cells and are not limited to components of whole blood. As such, blood cells include, for example, both white blood cells (WBCs) as well as rare cells, including CTCs.

The samples of this disclosure can each contain a plurality of cell populations and cell subpopulation that are distinguishable by methods well known in the art (e.g., FACS, immunohistochemistry). For example, a blood sample can contain populations of non-nucleated cells, such as erythrocytes (e.g., 4-5 million/μl) or platelets (150,000-400,000 cells/μl), and populations of nucleated cells such as WBCs (e.g., 4,500-10,000 cells/μl), CECs or CTCs (circulating tumor cells; e.g., 2-800 cells/). WBCs may contain cellular subpopulations of, e.g., neutrophils (2,500-8,000 cells/μl), lymphocytes (1,000-4,000 cells/μl), monocytes (100-700 cells/μl), eosinophils (50-500 cells/μl), basophils (25-100 cells/μl) and the like. The samples of this disclosure are non-enriched samples, i.e., they are not enriched for any specific population or subpopulation of nucleated cells. For example, non-enriched blood samples are not enriched for CTCs, WBC, B-cells, T-cells, NK-cells, monocytes, or the like.

In some embodiments, the sample is a biological sample, for example, a blood sample, obtained from a subject who has been diagnosed with prostate cancer based on tissue or liquid biopsy and/or surgery or clinical grounds. In some embodiments, the blood sample is obtained from a subject showing a clinical manifestation of prostate cancer advancing to CRPC, including without limitation, rising PSA levels prior to diagnosis, after initial surgery or radiation, or despite hormone therapy. In some embodiments, the sample is obtained from a subject who has been on hormone therapy or who has had a bilateral orchiectomy and whose testosterone levels have dropped to less than 50 ng/dl, and who shows evidence of disease progression in the form of rising PSA levels or bone or soft tissue metastases. In some cases, the sample is obtained from a subject who has been undergoing primary hormone therapies, which are the LHRH agonists, for example, leuprolide (Lupron) or goserelin (Zoladex). In other embodiments, the biological sample is obtained from a healthy subject or a subject deemed to be at high risk for prostate cancer and/or metastasis of existing prostate cancer based on art known clinically established criteria including, for example, age, race, family and history.

The methods of the invention further allow for resistance monitoring of prostate cancer patients by enabling detection of an emergence of CRPC in a patient afflicted with prostate cancer based on the correlation of phenotypic and genomic heterogeneity disclosed herein. The rapid evolution of drug therapies in prostate cancer has vastly improved upon the use of docetaxel since its pivotal US Food and Drug Administration (FDA) approval in 2004 and has brought about a new era where progress has been made beyond the use of androgen deprivation therapy (ADT) with the addition of novel hormonal agents, immunotherapy, second-line chemotherapy as well as radiopharmaceuticals. The choice of sequencing currently relies on patient profiles, whether symptoms of metastatic disease exist or not. While survival outcomes are undeniably improved with the use of these therapies, disease will ultimately progress on each regimen.

Androgens in the form of testosterone or the more potent dihydrotestosterone (DHT) have been well-defined drivers of progression of prostate cancer and differentiation of the prostate gland. As such, the backbone of treatment for advanced prostate cancers was established decades ago when castration in the form of surgical orchiectomy achieved significant prostate tumor regression. Since then, substitution to chemical castration has been employed mostly due to patient preference. ADT has therefore become the standard systemic treatment for locally advanced or metastatic prostate cancer. While ADT is almost always effective in most patients, disease progression to castration resistance inevitably occurs. It is now increasingly recognized that the androgen receptor (AR) remains overexpressed despite seemingly castrate levels of testosterone, since alternative receptors may activate the AR or other target genes may help perpetuate the castrate-resistant phenotype, hence the term “castration-resistance” has become widely adopted in the literature. The enhanced understanding of the role of these androgens in stimulating the growth of prostate cancer has led to the development and approval of a newer generation anti-androgen hormonal therapy drugs such as Zytiga (arbiterone), which blocks androgen production, and Xtandi (enzalutamide), an androgen receptor (AR) inhibitor. As described herein, the methods of the invention make it possible to tailor treatments more precisely and effectively and further allow for resistance monitoring of a prostate cancer patients based on the correlation of phenotypic and genomic heterogeneity disclosed herein.

In some embodiments of the methods disclosed herein, the patient is undergoing hormone treatment. In certain embodiments, the hormone treatment is primary ADT. In additional embodiments, the increase in the prevalence of the CTC population associated with CRPC predicts resistance to primary ADT and informs a subsequent decision to initiate secondary hormone treatment and/or to initiate cytotoxic therapy. In some embodiments, the subsequent treatment decision is a first “secondary” hormone therapy, such as antiandrogens and ketoconazole, which are options for nonmetastatic CRPC. In other embodiments, the subsequent treatment decision is a second-generation antiandrogen such as Enzalutamide (Xtandi), which is more potent than first-generation antiandrogens because of its ability to block nuclear translocation of AR and approved for use in mCRPC, or abiraterone (Zytiga), which is a potent androgen synthesis inhibitor. In some embodiments, the subsequent treatment decision is cytotoxic chemotherapy with a platinum-based regimen, for example and without limitation, docetaxel (Taxotere®), mitoxantronepaclitaxel (Taxol®) and cabazitaxel.

In some embodiments, the methods can further encompass individual patient risk factors, clinical, biopsy or imaging data, which includes any form of imaging modality known and used in the art, for example and without limitation, by X-ray computed tomography (CT), ultrasound, positron emission tomography (PET), electrical impedance tomography and magnetic resonance (MRI). It is understood that one skilled in the art can select an imaging modality based on a variety of art known criteria. Additionally, the methods disclosed herein, can optionally encompass one or more one or more individual risk factors that can be selected from the group consisting of, for example, age, race, family history, clinical history and/or data.

Risk factors for CRPC in the context of clinical data further include, for example, include PSA, bone turnover markers, bone pain, bone scans. In those cases, biopsies can be performed to confirm or rule out mCRPC and methods for detecting mCRPC in a patient afflicted with prostate cancer can further take encompass as a risk factor the resultant biopsy data. It is understood that one skilled in the art can select additional individual risk factors based on a variety of art known criteria. As described herein, the methods of the invention can encompass one or more individual risk factors. Accordingly, biomarkers can include, without limitation, imaging data, clinical data, biopsy data, and individual risk factors. As described herein, biomarkers also can include, but are not limited to, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof (e.g., glycoproteins, ribonucleoproteins, lipoproteins) as well as portions or fragments of a biological molecule.

Direct analysis of CTCs according to the methods of the invention can include both morphological features and immunofluorescent features. As will be understood by those skilled in the art, biomarkers can include a biological molecule, or a fragment of a biological molecule, the change and/or the detection of which can be correlated, individually or combined with other measurable features, with mCRPC. CTCs, which can be present a single cells or in clusters of CTCs, are often epithelial cells shed from solid tumors and are present in very low concentrations in the circulation of subjects. Accordingly, detection of CTCs in a blood sample can be referred to as rare event detection. CTCs have an abundance of less than 1:1,000 in a blood cell population, e.g., an abundance of less than 1:5,000, 1:10,000, 1:30,000, 1:50:000, 1:100,000, 1:300,000, 1:500,000, or 1:1,000,000. In some embodiments, the a CTC has an abundance of 1:50:000 to 1:100,000 in the cell population.

The samples of this disclosure may be obtained by any means, including, e.g., by means of solid tissue biopsy or fluid biopsy (see, e.g., Marrinucci D. et al., 2012, Phys. Biol. 9 016003). Briefly, in particular embodiments, the process can encompass lysis and removal of the red blood cells in a 7.5 mL blood sample, deposition of the remaining nucleated cells on specialized microscope slides, each of which accommodates the equivalent of roughly 0.5 mL of whole blood. A blood sample may be extracted from any source known to include blood cells or components thereof, such as venous, arterial, peripheral, tissue, cord, and the like. The samples may be processed using well known and routine clinical methods (e.g., procedures for drawing and processing whole blood). In some embodiments, a blood sample is drawn into anti-coagulent blood collection tubes (BCT), which may contain EDTA or Streck Cell-Free DNA™. In other embodiments, a blood sample is drawn into CellSave® tubes (Veridex). A blood sample may further be stored for up to 12 hours, 24 hours, 36 hours, 48 hours, or 60 hours before further processing.

In some embodiments, the methods of this disclosure comprise an initial step of obtaining a white blood cell (WBC) count for the blood sample. In certain embodiments, the WBC count may be obtained by using a HemoCue® WBC device (Hemocue, Ängelholm, Sweden). In some embodiments, the WBC count is used to determine the amount of blood required to plate a consistent loading volume of nucleated cells per slide and to calculate back the equivalent of CTCs per blood volume.

In some embodiments, the methods of this disclosure comprise an initial step of lysing erythrocytes in the blood sample. In some embodiments, the erythrocytes are lysed, e.g., by adding an ammonium chloride solution to the blood sample. In certain embodiments, a blood sample is subjected to centrifugation following erythrocyte lysis and nucleated cells are resuspended, e.g., in a PBS solution.

In some embodiments, nucleated cells from a sample, such as a blood sample, are deposited as a monolayer on a planar support. The planar support may be of any material, e.g., any fluorescently clear material, any material conducive to cell attachment, any material conducive to the easy removal of cell debris, any material having a thickness of <100 μm. In some embodiments, the material is a film. In some embodiments the material is a glass slide. In certain embodiments, the method encompasses an initial step of depositing nucleated cells from the blood sample as a monolayer on a glass slide. The glass slide can be coated to allow maximal retention of live cells (See, e.g., Marrinucci D. et al., 2012, Phys. Biol. 9 016003). In some embodiments, about 0.5 million, 1 million, 1.5 million, 2 million, 2.5 million, 3 million, 3.5 million, 4 million, 4.5 million, or 5 million nucleated cells are deposited onto the glass slide. In some embodiments, the methods of this disclosure comprise depositing about 3 million cells onto a glass slide. In additional embodiments, the methods of this disclosure comprise depositing between about 2 million and about 3 million cells onto the glass slide. In some embodiments, the glass slide and immobilized cellular samples are available for further processing or experimentation after the methods of this disclosure have been completed.

In some embodiments, the methods of this disclosure comprise an initial step of identifying nucleated cells in the non-enriched blood sample. In some embodiments, the nucleated cells are identified with a fluorescent stain. In certain embodiments, the fluorescent stain comprises a nucleic acid specific stain. In certain embodiments, the fluorescent stain is diamidino-2-phenylindole (DAPI). In some embodiments, immunofluorescent staining of nucleated cells comprises pan cytokeratin (CK), cluster of differentiation (CD) 45 and DAPI. In some embodiments further described herein, CTCs comprise distinct immunofluorescent staining from surrounding nucleated cells. In some embodiments, the distinct immunofluorescent staining of CTCs comprises DAPI (+), CK (+) and CD 45 (−). In some embodiments, the identification of CTCs further comprises comparing the intensity of pan cytokeratin fluorescent staining to surrounding nucleated cells. In some embodiments, the CTCs are CK− CTCs, that are identified as CTC based on other characteristics. As described herein, CTCs detected in the methods of the invention encompass traditional CTCs, cytokeratin negative (CK⁻) CTCs, small CTCs, and CTC clusters. In some embodiments, the CTC detection and analysis is accomplished by fluorescent scanning microscopy to detect immunofluorescent staining of nucleated cells in a blood sample. Marrinucci D. et al., 2012, Phys. Biol. 9 016003).

In particular embodiments, all nucleated cells are retained and immunofluorescently stained with monoclonal antibodies targeting cytokeratin (CK), an intermediate filament found exclusively in epithelial cells, a pan leukocyte specific antibody targeting the common leukocyte antigen CD45, and a nuclear stain, DAPI. The nucleated blood cells can be imaged in multiple fluorescent channels to produce high quality and high resolution digital images that retain fine cytologic details of nuclear contour and cytoplasmic distribution. While the surrounding WBCs can be identified with the pan leukocyte specific antibody targeting CD45, traditional CTCs can be identified, for example, as DAPI (+), CK (+) and CD 45 (−). In the methods described herein, the CTCs comprise distinct immunofluorescent staining from surrounding nucleated cells.

As described herein, CTCs encompass traditional CTCs, also referred to as high definition CTCs (HD-CTCs). Traditional CTCs are CK positive, CD45 negative, contain an intact DAPI positive nucleus without identifiable apoptotic changes or a disrupted appearance, and are morphologically distinct from surrounding white blood cells (WBCs). DAPI (+), CK (+) and CD45 (−) intensities can be categorized as measurable features during CTC enumeration as previously described. Nieva et al., Phys Biol 9:016004 (2012). The enrichment-free, direct analysis employed by the methods disclosed herein results in high sensitivity and high specificity, while adding high definition cytomorphology to enable detailed morphologic characterization of a CTC population known to be heterogeneous. In some embodiments, the phenotypic characteristics of a CTC detected in the methods of the invention comprise one or more of the group consisting of nucleus size, nucleus shape, presence of holes in nucleus, cell size, cell shape and nuclear to cytoplasmic ratio, nuclear detail, nuclear contour, prevalence of nucleoli, quality of cytoplasm and quantity of cytoplasm.

All patents and publications mentioned in this specification are herein incorporated by reference to the same extent as if each independent patent and publication was specifically and individually indicated to be incorporated by reference.

The following examples are provided by way of illustration, not limitation.

### EXAMPLE

### Example 1

### Intra-Patient Genomic Heterogeneity of Single Circulating Tumor Cells (CTCs) Associated to Phenotypic CTC Heterogeneity in Metastatic Castrate Resistant Prostate Cancer (mCRPC)

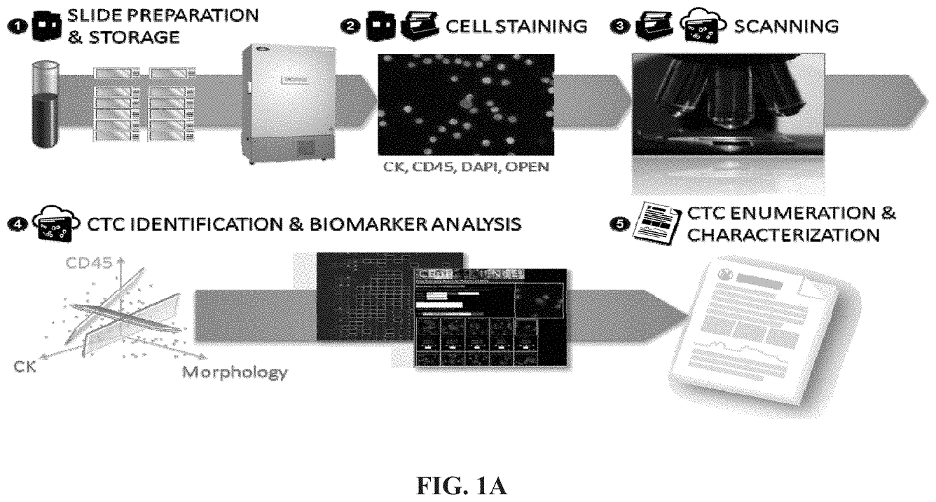

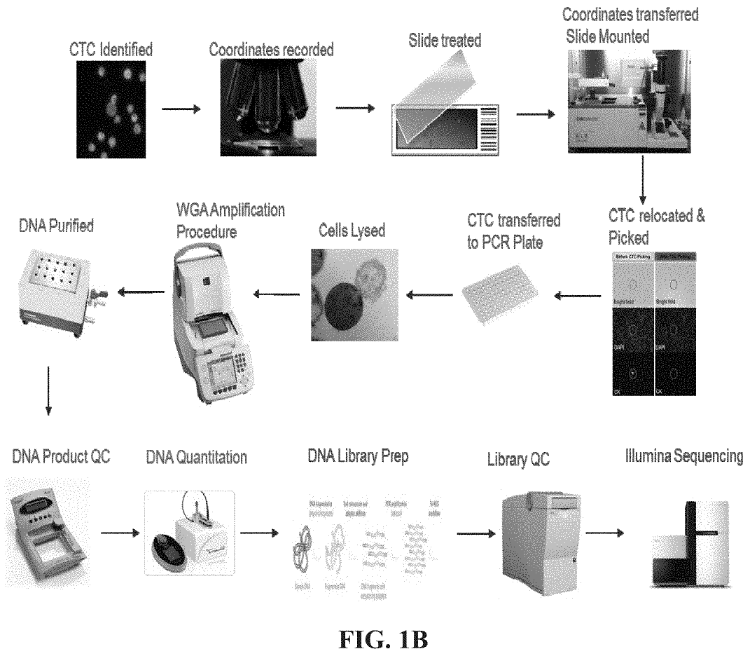

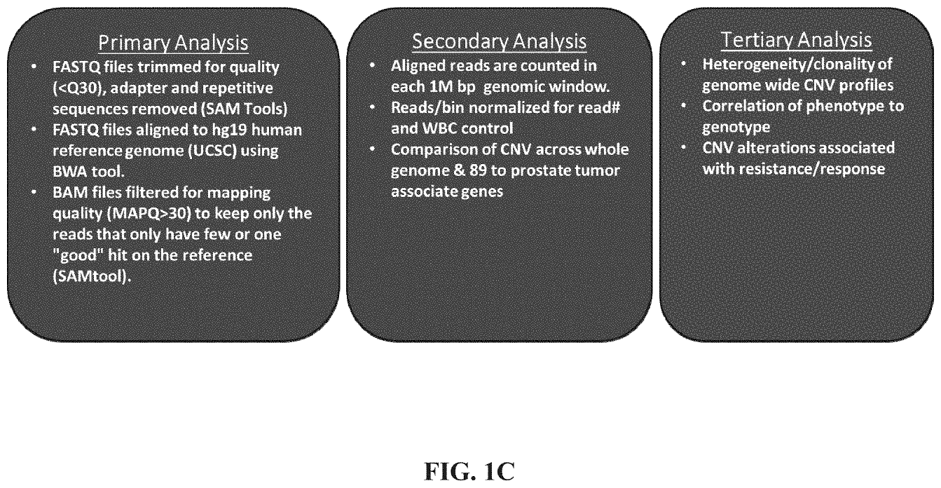

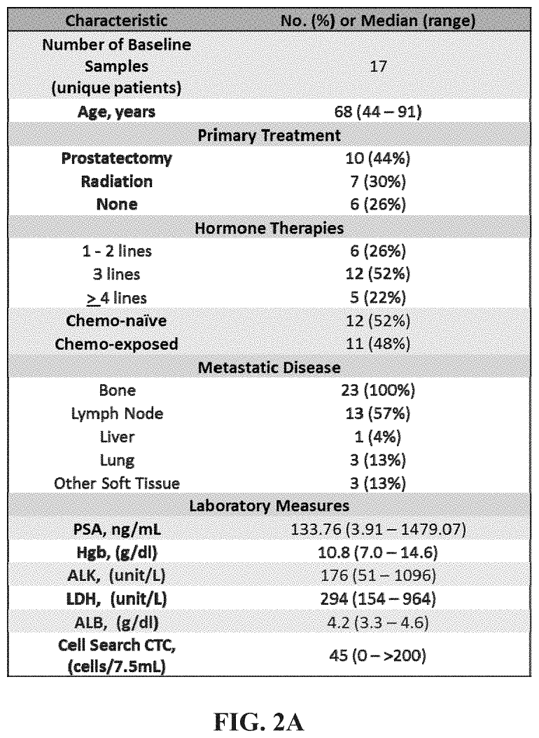

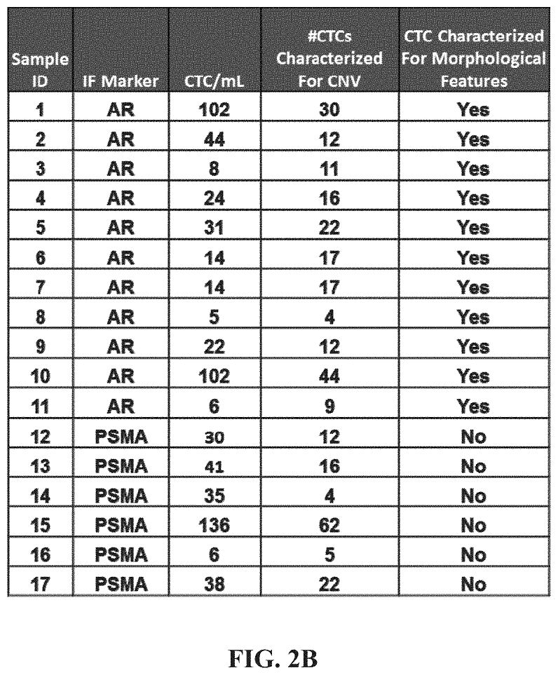

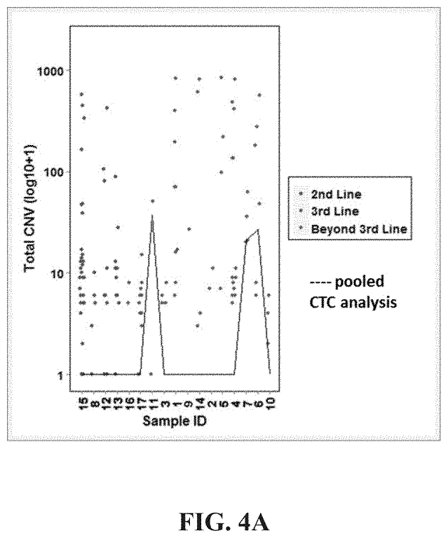

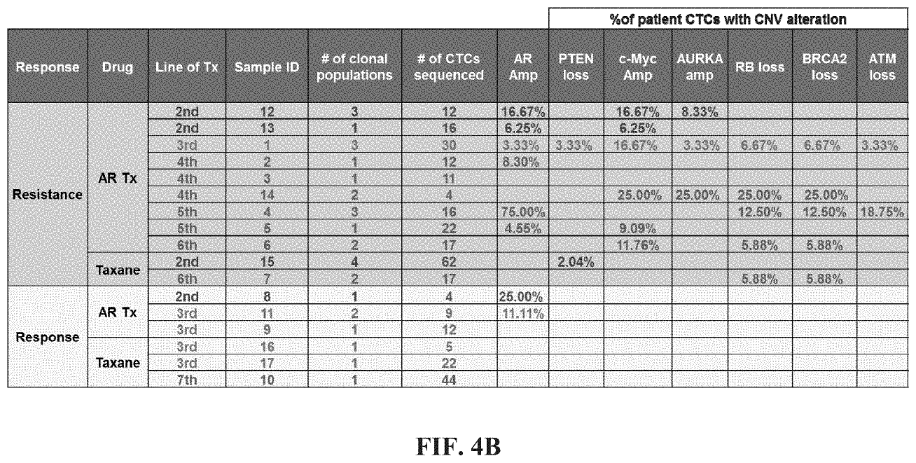

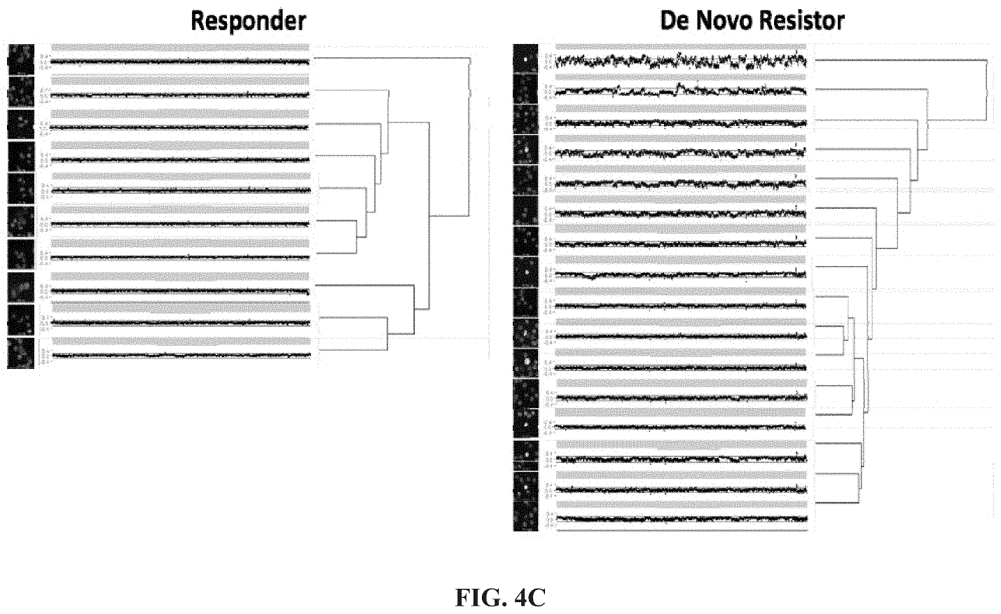

Example 1 demonstrates the existence of intra-patient genomic heterogeneity of single circulating tumor cells (CTCs) associated to phenotypic CTC heterogeneity in metastatic castrate resistant prostate cancer (mCRPC) as shown in FIGS. 1 to 5 and accompanying brief description of the drawings, supra.

1. A method for correlating intra-patient genomic heterogeneity of single CTCs with phenotypic heterogeneity in a prostate cancer (PCa) patient comprising:
(a) performing a direct analysis comprising immunofluorescent staining and morphological characterization of nucleated cells in a blood sample obtained from the patient to identify and enumerate circulating tumor cells (CTC);
(b) isolating the CTCs from said sample;
(c) individually characterizing genomic alterations and phenotypic features to generate a profile for each of the CTCs, and
(d) correlating genomic heterogeneity of single CTCs with phenotypic heterogeneity in the PCa patient.

2. A method for correlating intra-patient genomic heterogeneity of single CTCs with phenotypic heterogeneity in each of a population of prostate cancer (PCa) patients comprising:
(a) performing a direct analysis comprising immunofluorescent staining and morphological characterization of nucleated cells in a blood sample obtained from the patient to identify and enumerate circulating tumor cells (CTC);
(b) isolating the CTCs from said sample;
(c) individually characterizing genomic alterations and phenotypic features to generate a profile for each of the CTCs;
(d) correlating individual genomic heterogeneity of single CTCs with phenotypic heterogeneity in each of the population of PCa patients, and
(e) analyzing said correlations of individual genomic heterogeneity of single CTCs with phenotypic heterogeneity across the population of PCa patients to identify a universal correlation of individual genomic heterogeneity of single CTCs with phenotypic heterogeneity.

3. The method of claim 1, wherein said population of prostate cancer are similarly situated with regard to one or more patient demographics.

4. The method of claim 1, wherein said demographics comprise therapy or line of therapy.

5. The method of claim 4, wherein therapy is hormone directed therapy or chemotherapy.

6. The method of claim 5, wherein said hormone directed therapy comprises Androgen Deprivation Therapy (ADT).

7. The method of claim 6, wherein said ADT is a second line hormonal therapy.

8. The method of claim 7, wherein said second line hormonal therapy blocks synthesis of androgen or inhibits Androgen Receptor (AR).

9. The method of claim 8, wherein said second line hormonal therapy is selected from the group consisting of abiraterone acetate, ketoconazole and aminoglutethimide.

10. The method of claim 5, wherein said chemotherapy is taxane therapy.

11. The method of claim 1, wherein the immunofluorescent staining of nucleated cells comprises pan cytokeratin, cluster of differentiation (CD) 45, diamidino-2-phenylindole (DAPI) and androgen receptor (AR).

12. The method of claim 1, wherein said morphological characterization comprises determination of one or more of the group consisting of nucleus size, nucleus shape, presence of holes in nucleus, cell size, cell shape and nuclear to cytoplasmic ratio, nuclear detail, nuclear contour, prevalence of nucleoli, quality of cytoplasm and quantity of cytoplasm.

13. The method of claim 1, wherein said phenotypic features are selected from the group listed in FIG. 3D.

14. The method of claim 1, wherein said genomic alterations are copy number variation (CNV) alterations.

15. The method of claim 14, wherein said CNV alterations are selected from the group listed in FIG. 3D.

16. The method of claim 2, wherein said universal correlation is used to identify a phenotypic profile that corresponds to a genotypic profile.

17. The method of claim 16, wherein identification of said phenotypic profile obviates the need for characterizing said genomic alterations.

18. The method of claim 17, wherein said phenotypic profile is capable of predicting emergence of resistant disease.

19. The method of claim 18, comprising resistance to hormone directed therapy or chemotherapy.

20. The method of claim 19, wherein said hormone directed therapy comprises Androgen Deprivation Therapy (ADT).

21.-22. (canceled)
